(19) United States
(12) Patent Application Publication
(10) Pub. No.: US 2026/0076714 A1
(43) Pub. Date: Mar. 19, 2026

(54) NON-OCCLUDING INTRODUCER SHEATH WITH EXPANDABLE DISTAL TIP ANCHOR

(71) Applicant: BOSTON SCIENTIFIC SCIMED, INC., Maple Grove, MN (US)

(72) Inventors: Daniel Presteng, Corcoran, MN (US); Jeffrey Schneider, Maple Grove, MN (US); Zachary Kumor, Plymouth, MI (US); Benjamin Jerome Bidne, Loretto, MN (US); Lauren Ann Sandy, Minneapolis, MN (US)

(73) Assignee: BOSTON SCIENTIFIC SCIMED, INC., Maple Grove, MN (US)

(21) Appl. No.: 19/323,325

(22) Filed: Sep. 9, 2025

Related U.S. Application Data
(60) Provisional application No. 63/694,406, filed on Sep. 13, 2024.

(51) Int. Cl. A61B 17/34; A61B 17/00

(52) U.S. Cl. CPC A61B 17/3423; A61B 2017/00367; A61B 2017/3484; A61B 2017/3492

(57) ABSTRACT

An introducer sheath for use in delivering a device includes a hub, an elongate shaft including a proximal end region connected to the hub, a distal end region and a lumen extending therethrough. Further contemplated is a distal tip anchor that may be disposed proximate the distal end region. The distal tip anchor may have a collapsed configuration and an expanded configuration. The hub may include an actuation element operatively connected to the distal tip anchor such that actuation of the actuation element may deploy the distal tip anchor into the expanded configuration.

### CROSS REFERENCE TO RELATED APPLICATIONS

The present application claims the benefit of U.S. Provisional Patent Application Ser. No. 63/694,406, filed Sep. 13, 2024, the disclosure of which is incorporated herein by reference.

### TECHNICAL FIELD

The present disclosure relates to introducer sheaths for use in the intravascular delivery of medical devices. More particularly, the present disclosure relates to the use of an introducer sheath including an expandable distal anchor to improve and/or maintain vessel patency and vessel perfusion during a medical procedure.

### BACKGROUND

In various procedures, for example procedures for intravascularly delivering a medical device, such as a percutaneous device, an introducer sheath is introduced into a blood vessel, for example a femoral artery, and the medical device and/or tools may be inserted through the introducer sheath. For the delivery of certain medical devices, the introducer sheath may be sized with a diameter that substantially occludes the lumen of the blood vessel (e.g., is approximately equal to or greater than the diameter of the blood vessel). Insertion of an introducer sheath with a large diameter may cause damage to and/or tear the blood vessel, reduce blood flow therethrough, and/or create difficulties in sealing the vessel entry site.

It is further known in the art that during a medical procedure, the delivery and passage of medical devices through an introducer sheath may result in varying degrees of occlusion within the blood vessel. Large bore therapy devices with large distal sections, such as introducer sheaths, may cause limb ischemia (i.e., reducing blood flow through a vessel and affecting numbness in an extremity and/or limb of a patient) with extended periods of use.

Limb ischemia can escalate to cell and tissue death if blood flow is not reestablished to the affected area rapidly. Therefore, an unmet need exists in the art for an introducer sheath that improves the passage and effective use of large bore therapy devices while preventing blood flow occlusion through patient vasculature during medical procedures.

### BRIEF SUMMARY

This disclosure provides design, material, manufacturing method, and use alternatives for non-occluding introducer sheaths and the like. An example non-occluding introducer sheath of the present disclosure may include a hub and an elongate shaft. The elongate shaft may include a proximal end region connected to the hub, while also including a distal end region and a lumen extending therethrough. A distal tip anchor may be disposed proximate the distal end region. The distal tip anchor may include an expandable framework having a collapsed configuration and an expanded configuration. The hub may include an actuation element operatively connected to the distal tip anchor. Actuation of the actuation element may deploy the distal tip anchor into the expanded configuration.

Alternatively or additionally to any of the examples disclosed herein, the distal tip anchor may be contained within the elongate shaft in the collapsed configuration.

Alternatively or additionally to any of the examples disclosed herein, the distal tip anchor may be collapsed along an exterior of the elongate shaft in the collapsed configuration.

Alternatively or additionally to any of the examples disclosed herein, the distal tip anchor may include an expandable framework.

Alternatively or additionally to any of the examples disclosed herein, the distal tip anchor may further include a membrane affixed to the expandable framework.

Alternatively or additionally to any of the examples disclosed herein, the expandable framework may include a spiral member configured to expand radially outward from a distal tip of the elongate shaft upon actuation of the actuation element.

Alternatively or additionally to any of the examples disclosed herein, the expandable framework may include a plurality of struts. The plurality of struts may be adapted to expand radially outward from a distal tip of the elongate shaft upon actuation of the actuation element.

Alternatively or additionally to any of the examples disclosed herein, the expandable framework may include a pair of opposing beams, the pair of opposing beams configured to expand radially outward from the distal tip of the elongate shaft upon actuation of the actuation element.

Alternatively or additionally to any of the examples disclosed herein, the introducer sheath assembly may further include a proximal anchor that may be advanceable along an exterior of the shaft.

Alternatively or additionally to any of the examples disclosed herein, the proximal anchor may be advanceable along a threaded exterior region of the elongate shaft.

Alternatively or additionally to any of the examples disclosed herein, the proximal anchor may be adapted to be advanced distally along the exterior of the elongate shaft to trap body tissue between the proximal anchor and the distal tip anchor.

Alternatively or additionally to any of the examples disclosed herein, an introducer sheath assembly is disclosed and may include a hub. The introducer sheath of this and other examples may further include an elongate shaft. The elongate shaft may extend distally from the hub to a distal tip of the elongate shaft. The elongate shaft may also include a lumen extending therethrough. The introducer sheath assembly of this and other examples may include a distal tip anchor including an expandable framework having a collapsed configuration when positioned within the lumen and an expanded configuration when deployed from the distal tip of the elongate shaft. The hub may include an actuation element operatively connected to the distal tip anchor.

Alternatively or additionally to any of the examples disclosed herein, the introducer sheath assembly may include a proximal anchor advanceable along an exterior of the elongate shaft to trap body tissue between the proximal anchor and the distal tip anchor.

Alternatively or additionally to any of the examples disclosed herein, the proximal anchor may be advanceable along a threaded exterior region of the elongate shaft.

Alternatively or additionally to any of the examples disclosed herein, methods are further described and disclosed. In this and other examples, a method of maintaining vessel patency within a patient is provided. The method of this and other examples may include positioning an elongate shaft of an introducer sheath within a blood vessel of a patient, the elongate shaft including a proximal end region, a distal end region, and a lumen extending therebetween. The introducer sheath may further include a hub connected to the proximal end region of the elongate shaft, a distal tip anchor disposed proximate the distal end region, and an actuation assembly operatively connected to the distal tip anchor. The method of this and other examples may further include delivering a medical device through the elongate shaft of the introducer sheath into the blood vessel, actuating the actuation assembly to deploy the distal tip anchor from a collapsed configuration to an expanded configuration, positioning the distal tip anchor against an inner wall of the blood vessel with the distal tip anchor in the expanded configuration while the medical device remains in the blood vessel.

Alternatively or additionally to any of the examples disclosed herein, the methods described and disclosed herein may further include: advancing a proximal anchor along a threaded exterior region of the elongate shaft to trap body tissue between the proximal anchor and the distal tip anchor.

Alternatively or additionally to any of the examples disclosed herein, the methods described and disclosed herein may further include: breaking away the proximal end region from the distal end region by applying an outside force to a breakaway feature disposed between the proximal region and the distal end region.

Alternatively or additionally to any of the examples disclosed herein, the methods described and disclosed herein may further include the provision of an expandable framework included with and/or within the distal tip anchor.

Alternatively or additionally to any of the examples disclosed herein, the methods described and disclosed herein may further include the provision of a membrane affixed to the expandable framework.

Alternatively or additionally to any of the examples disclosed herein, the methods described and disclosed herein may further include, in the positioning step, positioning the distal tip anchor against an inner wall of the blood vessel with the distal tip anchor in the expanded configuration while the medical device remains within the blood vessel and further including the step of backing a length of the introducer sheath out of the blood vessel while the medical device remains within the blood vessel and forming a seal against an interior wall of the blood vessel with the distal tip anchor in the expanded configuration.

While multiple examples and embodiments are disclosed, still other examples and embodiments of the present disclosure will become apparent to those skilled in the art from the following detailed description, which shows and describes illustrative embodiments and examples of the present disclosure. Accordingly, the drawings and detailed description are to be regarded as illustrative in nature and not restrictive.

### BRIEF DESCRIPTION OF THE SEVERAL VIEWS OF THE DRAWINGS

The drawings illustrate the design and utility of preferred embodiments of the present disclosure. It should be noted that the figures are not drawn to scale and that elements of similar structures or functions are represented by like reference numerals throughout the figures. In order to better appreciate how the above-recited and other advantages and objects of the present disclosure are obtained, a more particular description of the present disclosure briefly described above will be rendered by reference to specific embodiments thereof, which are illustrated in the accompanying drawings. Understanding that these drawings depict only typical embodiments of the disclosure and are not therefore to be considered limiting of its scope, the present disclosure will be described and explained with additional specificity and detail through the use of the accompanying drawings, in which:

FIG. 1 illustrates a side view of an introducer sheath in a pre-deployment configuration.

FIG. 2A illustrates a side view of an introducer sheath after gaining percutaneous access to a blood vessel.

FIG. 2B illustrates a side view of the introducer sheath in an advanced configuration after gaining percutaneous access to the blood vessel.

FIG. 2C illustrates a side view of the introducer sheath as the distal tip is expanded and pulled back to the vessel wall of a patient or subject.

FIG. 2D illustrates a side view of the introducer sheath with movement of a proximal anchor depicted.

FIG. 2E illustrates an example hemostasis mechanism of the present disclosure.

FIG. 3A illustrates an end view of an expandable framework of a distal tip anchor contemplated by the present disclosure.

FIG. 3B illustrates an end view of an expandable framework of a distal tip anchor contemplated by the present disclosure.

FIG. 3C illustrates an end view of an expandable framework of a distal tip anchor contemplated by the present disclosure.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5). As used in this disclosure and appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some examples”, “other examples”, etc., indicate that the embodiment described may include one or more particular features, structures, and/or characteristics. However, such recitations do not necessarily mean that all examples include the particular features, structures, and/or characteristics. Additionally, when particular features, structures, and/or characteristics are described in connection with one embodiment, it should be understood that such features, structures, and/or characteristics may also be used connection with other examples whether or not explicitly described unless clearly stated to the contrary.

The following detailed description should be read with reference to the drawings in which similar elements in different drawings are numbered the same. The drawings, which are not necessarily to scale, depict illustrative examples and are not intended to limit the scope of the disclosure.

Various modifications and additions can be made to the exemplary embodiments discussed without departing from the scope of the present disclosure. For example, while embodiments and examples described above refer to particular features, the scope of the present disclosure also includes embodiments and examples having different combinations of features and embodiments and examples that do not include all of the described features herein.

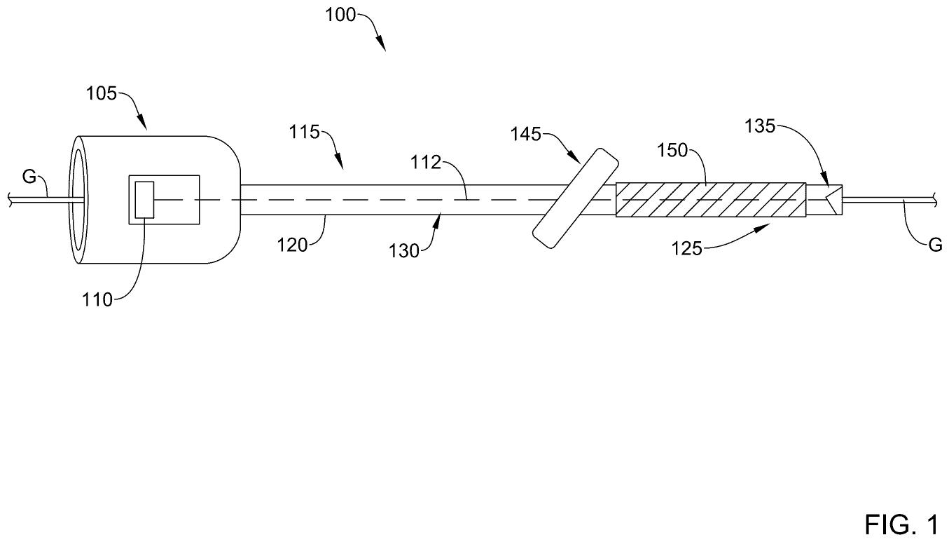

FIG. 1 illustrates a side view of an introducer sheath 100 in a pre-deployment configuration. The introducer sheath 100 may be advanceable along a guidewire (G) or other like component in the pre-deployment configuration for enabling access to patient or subject vasculature. For instance, an example introducer sheath 100 is shown with its distal tip anchor undeployed or contained, and thus yet to be deployed within a patient blood vessel or like anatomical structure. As shown in FIG. 1, the introducer sheath 100 includes an elongate shaft 115 including a proximal end region 120 extending to a proximal end of the elongate shaft 115 and a distal end region 125 extending to a distal end (i.e., distal tip) of the elongate shaft 115. The distal end region 125 may extend distally from the proximal end region 120 to the distal tip, in some instances. The proximal end region 120 may be operatively attached to a hub 105 with the elongate shaft 115 extending distally from the hub 105. The elongate shaft 115 may include a lumen 130 extending through the elongate shaft, such as from the hub 105 to the distal tip of the elongate shaft 115, for passage of a medical device therethrough.

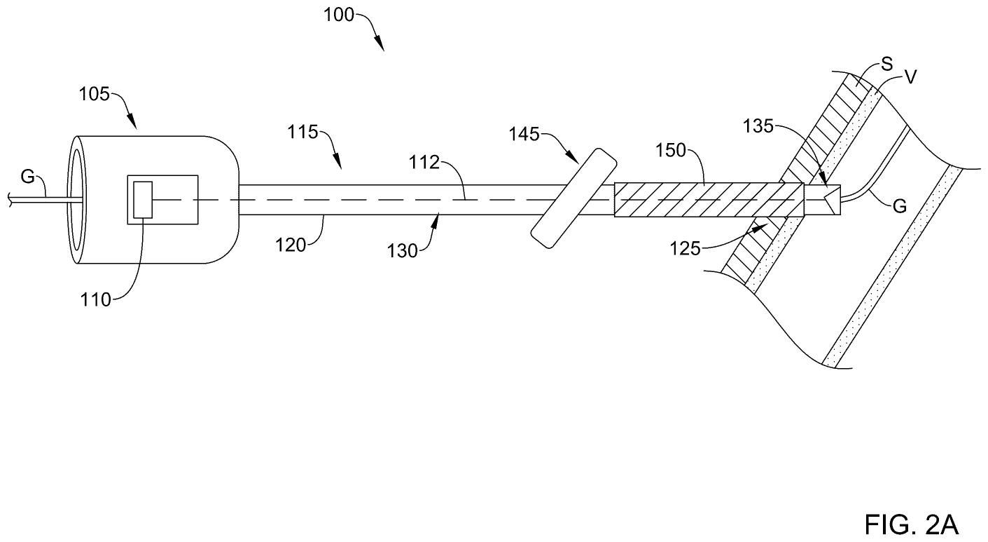

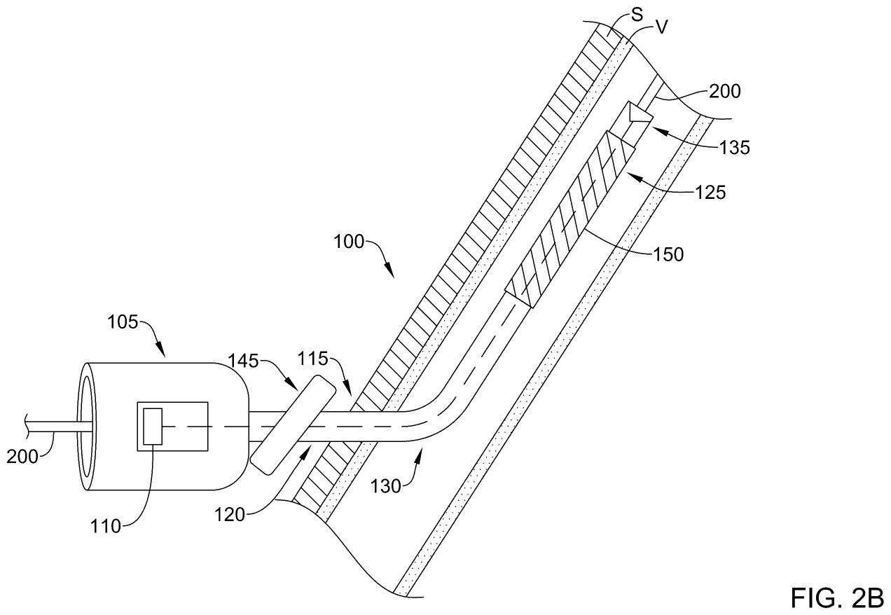

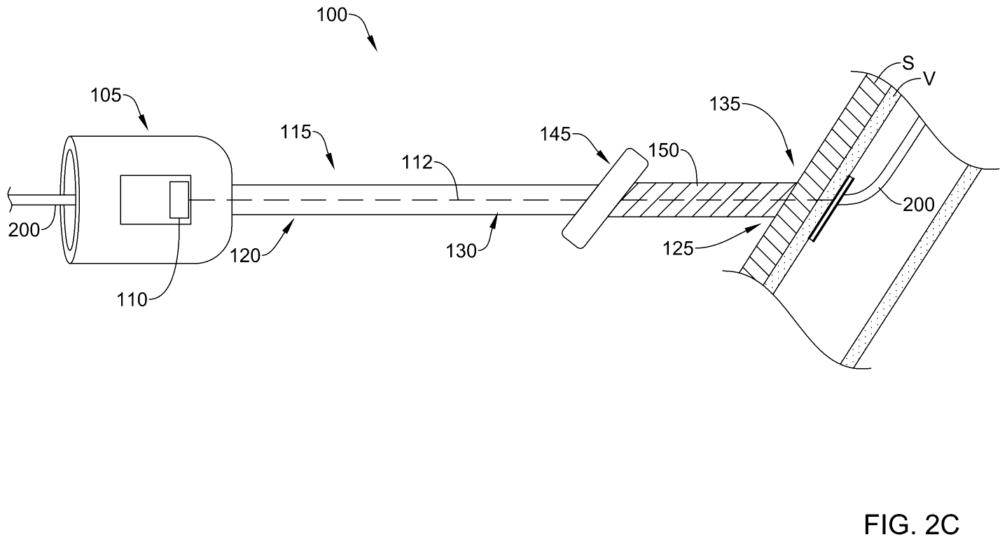

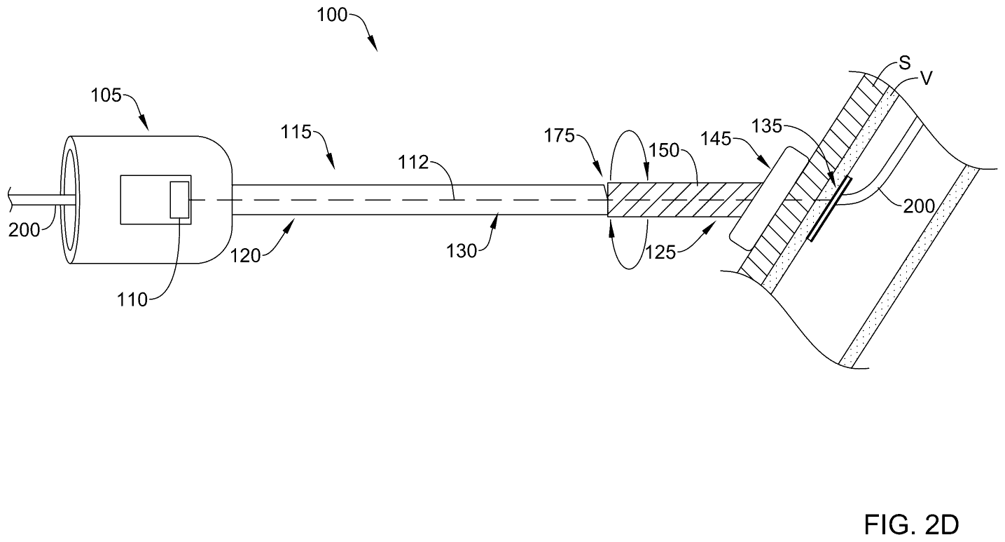

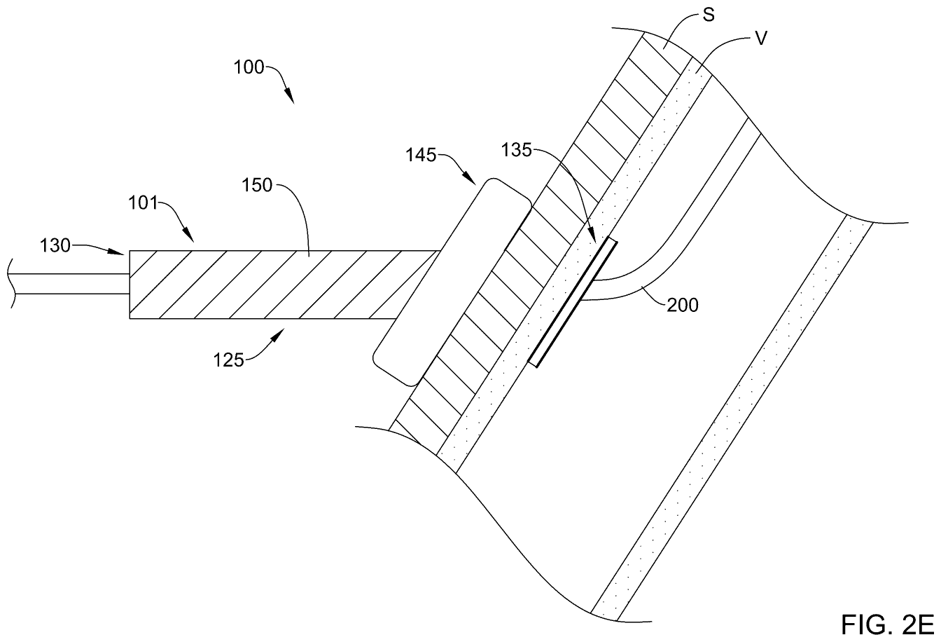

Introducer sheath 100 may further include a distal tip anchor 135 provided at the distal end region 125 proximate the distal tip of the elongate shaft 115. In this and other examples, the distal tip anchor 135 may have a collapsed configuration (shown in FIG. 1) and an expanded configuration (shown in FIG. 2C) as will be described further herein. As seen in FIG. 1, in some embodiments the distal tip anchor 135 may be contained within the lumen 130 of the elongate shaft in the collapsed configuration. In other embodiments, the distal tip anchor 135 may be collapsed against an exterior of the distal end region 125, such as contained between an inner surface of an outer sleeve and the exterior surface of the distal end region 125, in the collapsed configuration. Other arrangements of the distal tip anchor 135 in its collapsed configuration are also contemplated.

Hub 105 may include an actuation element 110. The actuation element 110 may act upon the distal tip anchor 135 when actuated, deploying the distal tip anchor 135 to an expanded configuration as will be described further herein. For example, the actuation element 110 may be actuatable by a user to deploy the distal tip anchor 135 distally from the lumen 130 of the elongate shaft 115, and thus actuate the distal tip anchor 135 to an expanded configuration. In other instances, the actuation element 110 may be actuatable to cause the distal tip anchor 135 to expand outward from the exterior of the elongate shaft 115 (e.g., by withdrawing an outer sleeve proximally to uncover the distal tip anchor 135). In some instances, the actuation element 110 is connected to the distal tip anchor 135 via a push/pull wire 112 extending along the elongate shaft 115 between the actuation element 110 and the distal tip anchor 135. Thus, movement of the actuation element 110 may be translated to movement of the distal tip anchor 135 through the push/pull wire 112. In some instances, the push/pull wire 112 may extend through the lumen 130 of the elongate shaft 115, along a separate lumen of the elongate shaft 115, or along an exterior of the elongate shaft 115, for example. In some embodiments, the actuation element 110 may include but is not limited to: a button, a knob, a lever, a pull wire, a pair of pull wires, a series of pull wires, a pneumatic actuator, an electronic actuator, a push-button actuator, a mechanical actuator, an electromechanical actuator, an electrochemical actuator, a pulley, a switch, a spool-and-line system (i.e. a reel), a piezoelectric actuator, any combination or permutation of the aforementioned, and/or the like.

Further shown in FIG. 1 is proximal anchor 145. Proximal anchor 145 may be movably disposed along any portion of the elongate shaft 115 of the introducer sheath 100, including but not limited to the proximal end region 120 of the elongate shaft 115. Proximal anchor 145 may be movably disposed such that proximal anchor 145 may translate, rotate, slide, or otherwise move along the elongate shaft 115 of the introducer sheath 100 in any direction (e.g., proximally and/or distally along the elongate shaft 115). In some instances, the proximal anchor 145 may engage with a threaded exterior region 150 of the elongate shaft 115. For instance, the proximal anchor 145 may include a threaded bore configured to threadably engage the threaded exterior region 150 of the elongate shaft 115. Thus, rotation of the proximal anchor 145 about the elongate shaft 115 (i.e., rotation of the proximal anchor 145 about the central longitudinal axis of the elongate shaft 115), in a first direction may advance the proximal anchor 145 distally along the threaded exterior region 150 of the elongate shaft 115 toward the distal tip anchor 135 and thus the distal tip of the elongate shaft 115, whereas rotation of the proximal anchor 145 about the elongate shaft 115 (i.e., rotation of the proximal anchor 145 about the central longitudinal axis of the elongate shaft 115), in a second, opposite direction may advance the proximal anchor 145 proximally along the threaded exterior region 150 of the elongate shaft 115 away from the distal tip anchor 135 and thus the distal tip of the elongate shaft 115. Proximal anchor 145 may take the form of a dial, a knob, a disc, a handle, a protrusion, a ring, an annular structure, a collar or any like structure. Alternatively or additionally, proximal anchor 145 may aid in forming a seal at a vessel site or other like anatomical structure by being moved (i.e., translated, rotated, shifted, etc.) along the elongate shaft 115 until it interfaces against a patient's skin at a venotomy or arteriotomy (e.g., vessel access site). From there the proximal anchor 145 may be further tightened against the patient's skin to trap body tissue between the proximal anchor 145 and the distal tip anchor 135. The proximal anchor 145 may be tightened by any means or method known in the art, including but not limited to: applying torque, twisting, snap-fitting, interference fitting, threading to or along a threaded coupling, coupling to a male-female coupling, coupling via a slotted coupling, or the like. Proximal anchor 145 may additionally be tightened and translated over an outer sleeve (not shown) as it is pressed against the patient's skin. The outer sleeve aiding in providing a thorough seal to the vessel access site or an access site of like anatomical structure.

Threaded exterior region 150 may be one or more helical threads (continuous or discontinuous) disposed along the distal end region 125 of the elongate shaft 115. Alternative designs and implementations of threads and threaded couplings are also contemplated. By non-limiting example, the threaded exterior region 150 may be formed (e.g., molded, cut, scored, etc.) in the elongate shaft 115, or the threaded exterior region 150 may be part of a separate sleeve, conduit or other device disposed about or around the elongate shaft 115, including but not limited to the distal end region 125 of the elongate shaft 115. Alternatively or additionally, the threads of the threaded exterior region 150 may be left-handed threads (i.e., counterclockwise threads), right-handed threads (clockwise threads), threads of consistent and/or constant pitch, threads of varying pitch, threads of consistent and/or constant width, threads of varying width and/or any combination or permutation of the aforementioned. It is additionally contemplated that the proximal anchor 145 may lock or otherwise engage with the distal end region 125 proximate the distal end region 125 of the elongate shaft 115 by engagement and/or locking means other than a threaded connection. In other words, the proximal anchor 145 may movably (i.e., translate, rotate, slide, etc.) lock or otherwise lockably engage with the distal end region 125 or near the distal end region 125 of the elongate shaft 115 by means including but not limited to: an interference fit, a friction fit, a male-female coupling, a slotted connection, a series of slotted connections, a keyed connection, a snap-fit connection, a twist lock, corrugations, interlocking grooves, interlocking ridges, interlocking corrugations, interlocking protrusions, interlocking projections, or any combination of the aforementioned or the like.

The distal tip anchor 135 may be positioned or positionable proximate to the distal end region 125 of the elongate shaft 115. By non-limiting examples, the distal tip anchor 135 may reside within the lumen 130 of the elongate shaft 115, outside the lumen 130 of the elongate shaft 115, within the interior of the introducer sheath 100, or exterior of the introducer sheath 100 in its collapsed, delivery configuration. The distal tip anchor 135 may be actuated to move to an expanded, deployed configuration within the vessel that the introducer sheath 100 is positioned within. In the expanded, deployed configuration, the distal tip anchor may 135 extend radially outward beyond the outer diameter of the elongate shaft 115.

Alternatively or additionally to this and other examples, the distal tip anchor 135 may be collapsed along an exterior of the elongate shaft 115 in the collapsed configuration. In some instances, an outer sheath (not shown) may be disposed over the distal end region 125 of the introducer sheath 100 and may surround, encapsulate, envelop or at least partially surround the distal tip anchor 135 in its collapsed configuration up until deployment into an expanded configuration. In this and other examples, when it is appropriate for the distal tip anchor 135 to be deployed into an expanded configuration (i.e., when the distal tip anchor resides inside a vessel and/or is ready to form an anchor and/or seal against the inside wall of a vessel), the outer sheath may be retracted proximally to uncover at least a portion of the distal tip anchor 135, such that the distal tip anchor 135 automatically deploys into an expanded configuration. This may be accomplished by actuation of an actuator, such as actuation element 110 to withdraw the outer sheath proximally, or may be accomplished by the material nature of the distal tip anchor 135.

In some embodiments, the distal tip anchor 135 and all associated components (including those which include expandable membrane 155) may be heat-set and radially biased in its radially expanded configuration such that the distal tip anchor 135 is collapsed (e.g., constrained, deformed or deflected) away from its natural, expanded configuration (i.e., in which the distal tip anchor 135 is in an unconstrained, equilibrium state) against the exterior wall of the introducer sheath 100, collapsed within the lumen 130 of the introducer sheath 100 and/or collapsed within the inner wall of the outer sheath. Therefore, when the distal tip anchor 135 is exposed outside of and/or not constrained by these aforementioned structures, the distal tip anchor 135 automatically expands radially outward given its material nature of being heat-set and radially biased. Further, the distal tip anchor 135 and all associated components may be heat-set and/or temperature-dependent, radially biased and deployed by actuation of actuation element 110 into a radially expanded configuration.

FIG. 2A depicts the example introducer sheath 100 after the introducer sheath 100 has gained percutaneous access through an incision in the skin S of a patient to a blood vessel V via a guidewire G. As shown in FIG. 2A, the introducer sheath 100 has gained percutaneous access to a blood vessel V and the distal tip anchor 135 resides in its collapsed configuration within the distal end region 125 of the elongate shaft 115 of the introducer sheath 100.

Continuing to FIG. 2B, the introducer sheath 100 is shown having gained deeper access into the vasculature with the elongate shaft 115 extending into the vessel V distally beyond the insertion site with the distal tip anchor 135 remaining in a collapsed configuration. In some instances, the distal end of the elongate shaft 115 may be advanced in the lumen of the blood vessel V a distance of 12.7 centimeters (cm) (5 inches) or more, 15.2 cm (6 inches) or more, 20.3 cm (8 inches) or more, or other distance, from the insertion site. A medical device 200, (e.g., a blood pump, or other medical device) may then be advanced through the lumen 130 of the elongate shaft 115 of the introducer sheath 100 into the blood vessel V and distally beyond the introducer sheath 100. The medical device 200 may be inserted through the hub 105 and passed through the lumen 130 of the elongate shaft 115 as the medical device 200 passes through the distal end region 125 of the introducer sheath 100 and into the patient or subject vasculature.

It is to be noted, that the distal tip anchor 135 may be deployed into its expanded configuration by action of the actuation element 110 at any time during a procedure, including while the introducer sheath 100 or at least a portion of the introducer sheath 100 resides within the vasculature of a patient or subject. In this and other examples, actuation of the actuation element 110 may push the distal tip anchor 135, stretch the distal tip anchor 135, bend the distal tip anchor 135, oscillate the distal tip anchor 135, tilt the distal tip anchor 135, and/or retract the distal tip anchor 135. Further, distal tip anchor 135 may be heat-set and radially biased to its expanded configuration. In other words, distal tip anchor 135 may be heat-set and radially biased such that the distal tip anchor 135 automatically assumes its expanded configuration once unconstrained by the introducer sheath 100 (e.g., extends distally beyond the distal tip of the introducer sheath 110 or is positioned beyond a distal tip of an outer sheath, as discussed above.

Continuing to FIG. 2C, once the medical device 200 has been advanced through the introducer sheath 100 to a remote location within the vasculature of the patient, distal tip anchor 135 may be deployed to the expanded configuration and the introducer sheath 100 may be withdrawn proximally until the distal tip anchor 135 contacts or engages the inner surface of the vessel V at the insertion site while the medical device 200 (not shown in FIG. 2C for clarity) still resides within the introducer sheath 100 and extends distally within the blood vessel V. It can be appreciated that the introducer sheath 100 may be retracted along a proximal retraction direction, which is any direction coinciding with the path of the introducer sheath 100 being withdrawn proximally from an accessed vessel. Thereby the distal tip anchor 135 may be deployed into an expanded configuration to engage, anchor and/or form a seal against an interior wall of the blood vessel V, as shown in FIG. 2C, as the distal tip anchor 135 and/or the distal end region 125 is pulled back, engaged with, and/or aligned flush to the interior wall of the vessel V. Accordingly, access to the vessel V may still be retained with the distal end region 125 of the elongate shaft 115 and/or the distal tip anchor 135 positioned within the lumen of the blood vessel V while the remainder of the introducer sheath 100 has been withdrawn from the blood vessel V. Furthermore, the medical device 200 may remain positioned in the blood vessel V, extending out the distal tip of the elongate shaft 115 while the distal tip anchor 135 is engaged with and/or anchored against the interior wall of the blood vessel V. By withdrawing the elongate shaft 115 until the distal tip anchor 135 is engaged with or anchored against the interior wall of the vessel V, the patency of the blood vessel V may be maintained, allowing blood flow through the vessel V while maintaining access to the blood vessel V with the introducer sheath 100.

To further aid in sealing the vessel access site, the proximal anchor 145 may be advanced distally along the elongate shaft 115, such as in the rotational manner depicted by the pair of directional arrows displayed in FIG. 2D. For instance, the proximal anchor 145 may be rotated to advance the proximal anchor 145 distally along the threaded region 150 of the elongate shaft 115. The proximal anchor 145 may be advanced distally until it abuts the patient's skin S, thereby trapping body tissue (including the skin S and wall of the blood vessel V) between the proximal anchor 145 and the distal tip anchor 135. Once sufficiently advanced distally, the proximal anchor 145 may be locked into a desired position to stabilize the introducer sheath 100 within the vessel V and vessel access site or within a like anatomical structure. This may be beneficial as it allows a user and/or practitioner to continue a medical procedure and/or treatment while keeping the introducer sheath 100 within the vessel in a non-occlusive manner to both protect the vessel walls and prevent health risks to the patient such as critical limb ischemia. This further reduces time of procedure and operating costs as less time is devoted to swapping introducer sheaths, attending to vascular occlusions and/or deleterious conditions, and other delays associated with lack of proper and enduring stabilization of introducer sheaths. As described herein, proximal anchor 145 may be advanced along the elongate shaft 115 by means other than torque or twisting. By non-limiting examples, the proximal anchor 145 may be advanced distally and/or retracted proximally along the elongate shaft 115 by translation, sliding, overcoming frictional resistance, by electronic actuation, by mechanical actuation, or the like.

Accordingly, as shown in FIG. 2D the introducer sheath 100 is shown in a deployed and retracted configuration, in which the introducer sheath 100 is pulled back to the vessel wall of a patient or subject and the distal tip is expanded and deployed. As shown in FIG. 2D, the distal tip anchor 135 is deployed in its expanded configuration through action of the actuation element 110. The distal tip anchor 135 and expandable framework 155 may be expanded against the interior wall of the blood vessel V to form a complete seal while allowing luminal access to the introducer sheath 100 during medical procedures. Furthermore, the proximal anchor 145 may be positioned in a fully locked position against the patient's or subject's skin S, thereby increasing the effectiveness of the seal in and around the introducer sheath 100 where it interfaces a patient or subject access site.

FIG. 2D further displays a breakaway feature 175 which may be provided with the introducer sheath 100 in some instances. Breakaway feature 175 is any structure that allows one portion of the elongate shaft 115 to be separated from another portion of the elongate shaft 115, typically along or proximate to the breakaway feature. By non-limiting examples, breakaway feature 175 may be a singular breakaway feature or include multiple breakaway features. Breakaway feature 175 may be in the form of a notch, a score mark, a groove, a perforation, a weaker material (i.e., material(s) weaker than adjacent and/or surrounding material(s)), a tearable material (i.e., a material that can be torn or broken away from adjacent and/or surrounding material), a splittable material, a heat-sensitive material, a cold-sensitive material, a tearable sheath (i.e. a sheath that can be torn or broken away from adjacent structure), a splittable sheath, a series of perforations, a series of notches, a series of score marks, a series of grooves, a series of weaker materials, a series of tearable materials, a series of splittable materials, a series of tearable sheaths, a series of splittable sheaths, or the like.

In some instances, as shown in FIG. 2E, the proximal portion of the introducer sheath 100 may be separated from the distal portion of the introducer sheath 100 at the breakaway feature 175, leaving the distal portion of the introducer sheath 100 at the access site. The remaining distal portion of the introducer sheath 100 may act as a hemostasis mechanism 101 at the access site, with the distal tip anchor 135 engaging and/or sealing the inner surface of the vessel V and the proximal anchor 145 engaging and/or sealing the incision at the insertion site. Hemostasis mechanism 101 may act to maintain a seal at an access site of a patient or subject such as an arterial access site through an arteriotomy. Hemostasis mechanism 101 may be activated or otherwise utilized after the proximal end region 120 of the introducer sheath 100 has been broken away from the distal end region 125 by use of breakaway feature 175. Breakaway feature 175 of this and other examples may be any breakaway feature described or disclosed herein. After breaking away the proximal end region 120 from the distal end region 125 via breakaway feature 175, the distal end region 125 may form a hemostasis mechanism 101 which acts as a blood seal or other like hemostasis device as it aids in forming a seal at the patient or subject access site, and may remain within the patient or subject for the duration of a medical procedure. An added benefit to the hemostasis mechanism 101 and this breakaway capability provided by the breakaway feature 175 is that a significant length of the introducer sheath (e.g., proximal length of introducer sheath) may be discarded to free up operational space in an operational and/or medical setting.

In the various embodiments described herein, the distal tip anchor 135 may include an expandable framework configured to expand outward beyond an outer diameter of the distal end region 125 of the elongate shaft 125 when deployed. The expandable framework may be flexible to move between a collapsed configuration to an expanded configuration, and may be biased to assume the expanded configuration when unconstrained. In some instances, the distal tip anchor 135 may additionally or alternatively include an expandable membrane attached to, coupled to, adhered to, bonded to, braided, or otherwise affixed to any, some or all portions of the distal tip anchor 135. Expandable membrane may take the form of a polymer mesh, a multi-polymer mesh, a wire mesh, an elastomer, an elastomeric mesh, an elastomeric web, a polymeric web, an elastomeric weave, a polymeric weave, an elastomeric balloon, a polymeric balloon, or other configuration. In some instances, the expandable membrane may function in coordination with the expandable framework to form a seal against the interior wall of a vessel while also preventing occlusion within the vessel during use of devices associated with the present disclosure.

In this and other examples, the expandable framework of the distal tip anchor 135 may include one or more arms, struts, projections, spirals, filaments, beams, ribbons, or other projecting structures or features. These aforementioned structures and features may expand radially outwardly from the introducer sheath 100, the introducer sheath lumen 130, and/or the distal end region 125 and may be heat-set and radially biased outwardly, and may be deployed into an expanded configuration by actuation of actuation element 110.

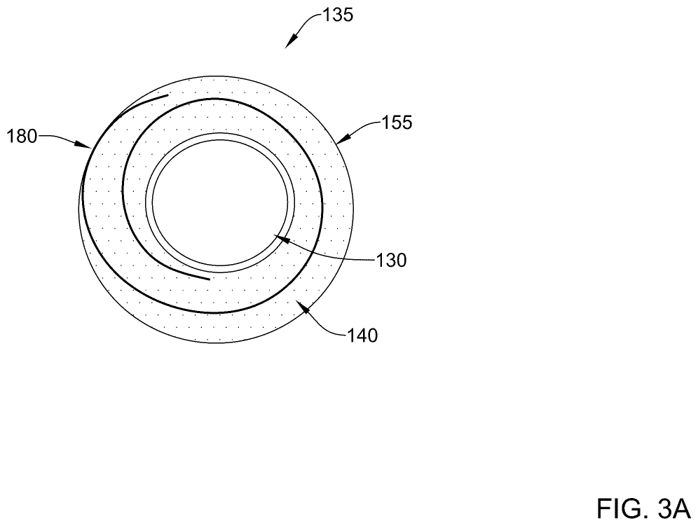

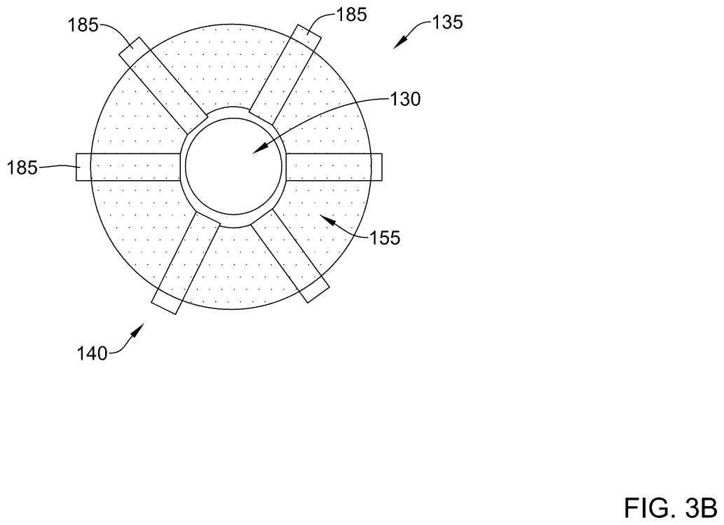

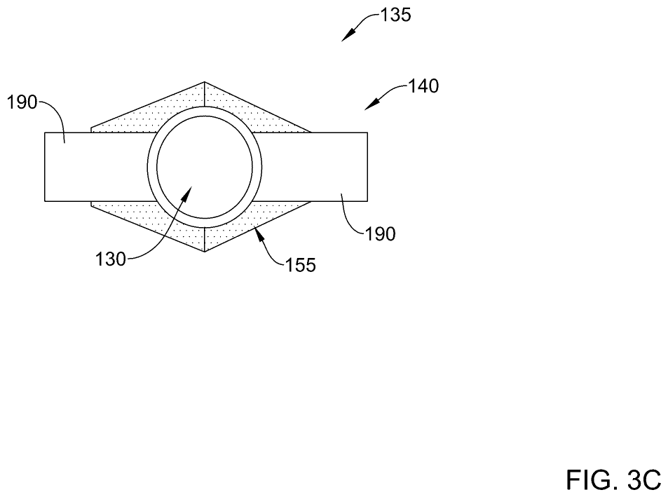

FIG. 3A illustrates an end view of an example distal tip anchor 135. As shown in FIG. 3A, distal tip anchor 135 may include an expandable framework 140 including a spiral member 180 or a plurality of spiral members. Spiral member 180 may be one or more, or a series of interconnected or continuous arms, struts and/or members that form a spiral shape and act to form an anchor and/or seal against the interior of a vessel wall or similar anatomical structure, and in some instances may do so in coordination with an expandable membrane 155 attached thereto. Alternatively or additionally, spiral member 180 may be a continuous or contiguous structure. The spiral member 180 may extend outwardly beyond the outer diameter of the distal end region 125 of the elongate shaft 115 when unconstrained, and deployed into the expanded configuration, as described above.

The expandable framework (e.g., the spiral member 180) may further include an expandable membrane 155 attached or otherwise coupled thereto. As described above, expandable membrane 155 may be formed by various materials and/or combinations of materials including but not limited to: polymers, elastomers, metals, alloys, plastics, thermoplastics, or thermopolymers (i.e. a polymer or combination of polymers that changes size and/or shape under the influence of certain temperatures). Additionally, spiral member 180 may be formed of any suitable material and/or combinations of materials including but not limited to: metals, alloys, plastics, thermoplastics, polymers or thermopolymers. Expandable membrane 155 may be attached or coupled to only one portion of the spiral member 180 or plurality of spiral members. In other non-limiting examples, expandable membrane 155 may be attached or coupled to only two portions of the spiral member 180 or plurality of spiral members. In yet other non-limiting examples, expandable membrane 155 may be attached or coupled to only three portions of the spiral member 180 or plurality of spiral members, only four portions of the spiral member 180 or plurality of spiral members, or five or more portions of the spiral member 180 or plurality of spiral members.

FIG. 3B illustrates an end view of an example distal tip anchor 135. As shown in FIG. 3B, distal tip anchor 135 may include an expandable framework 140 including one or more, or a plurality of struts 185. Struts 185 may include one strut, two struts, three struts, four struts, or five or more struts. Struts 185 may extend outwardly from lumen 130, outwardly from introducer sheath 100, radially outward from lumen 130, radially outward from introducer sheath 100, radially outward from the distal end region 125 or may have struts that alternate movement relative to other struts. In other words, struts 185 may extend outwardly beyond the outer diameter of the distal end region 125 of the elongate shaft 115 when unconstrained, and deployed into the expanded configuration, as described above. In some instances, struts 185 may selectively engage a vessel wall by deploying a preset number of struts less than the full number of struts present on the device, or may fully engage a vessel wall by deploying all struts present on the device.

Struts 185 may take the form of a ribbon or ribbons, and may extend from any one portion of the distal tip anchor 135 and/or expandable framework 140 to any other portion of the distal tip anchor 135 and/or expandable framework 140 and may include more than two attachment points. In this and other examples, the ribbon or ribbons may bow or buckle inward or outward to provide greater flexibility in forming a seal against a vessel wall or other like anatomical structure.

Struts 185 may further include an expandable membrane 155 attached or otherwise coupled thereto. Expandable membrane 155 may be attached or coupled to only one strut, one or more struts, two or more struts, three or more struts, four or more struts, or five or more struts. Alternatively or additionally, expandable membrane 155 may be attached or coupled to only one portion of a strut, only two portions of a strut, only three portions of a strut, four or more portions of a strut, or five or more portions of a strut. Alternatively or additionally, expandable membrane 155 may be attached or coupled to only one portion among the entirety of an array of struts (i.e., among all struts present on the device). In other non-limiting examples, expandable membrane 155 may be attached or coupled to only two portions among the entirety of an array of struts. In yet other non-limiting examples, expandable membrane 155 may be attached or coupled to only three portions among the entirety of an array of struts, only four portions among the entirety of an array of struts or five or more portions among the entirety of an array of struts.

FIG. 3C illustrates an end view of an example distal tip anchor 135. As shown in FIG. 3C, distal tip anchor 135 may include an expandable framework 140 including one or beams 190, such as one or more pairs of opposing beams 190. Beams 190 may extend outwardly beyond the outer diameter of the distal end region 125 of the elongate shaft 115 when unconstrained, and deployed into the expanded configuration, as described above. Beams 190 may be formed of any suitable material or combination of materials including but not limited to metals, alloys, plastics, thermoplastics, polymers, or thermopolymers.

Beams 190 may further include an expandable membrane 155 attached, affixed or otherwise coupled thereto. Expandable membrane 155 may be attached or coupled to only one beam, one or more beams, two or more beams, three or more beams, four or more beams, or five or more beams. Alternatively or additionally, expandable membrane 155 may be attached or coupled to only one portion of a beam, only two portions of a beam, only three portions of a beam, four or more portions of a beam, or five or more portions of a beam. Alternatively or additionally, expandable membrane 155 may be attached or coupled to only one portion among the entirety of an array of beams (i.e., among all beams present on the device). In other non-limiting examples, expandable membrane 155 may be attached or coupled to only two portions among the entirety of an array of beams. In yet other non-limiting examples, expandable membrane 155 may be attached or coupled to only three portions among the entirety of an array of beams, only four portions among the entirety of an array of beams or five or more portions among the entirety of an array of beams.

The expandable framework 140 and/or the expandable membrane 155 of the distal tip anchor 135 may conform to several shapes and geometries in its expanded, deployed configuration, including but not limited to: ellipsoidal, substantially ellipsoidal, spherical, substantially spherical, disc-shaped, substantially disc-shaped. In some instances, the expandable membrane 155 may be adhered to the expandable framework 140. In other instances, the expandable membrane 155 may be molded to the expandable framework 140, such that the expandable framework 140 is partially embedded in or encapsulated by the expandable membrane 155, or the expandable membrane 155 may be molded to the expandable framework 140, such that the expandable framework 140 is fully embedded in or encapsulated by the expandable membrane 155. In other instances, the expandable membrane 155 may be otherwise attached to the expandable framework 140, such as with fasteners, heat bonding, sutures, or the like. In some instances, the distal tip anchor 135 may include a collar or ring, and the expandable framework 140 and/or the expandable membrane 155 may be coupled to and/or extend from the collar. In some instances, the collar may be provided at a proximal end of the distal tip anchor 135 such that the distal tip anchor 135 may be fixed to the distal end of the push/pull wire 112 via the collar.

Alternatively or additionally, the expandable framework 140 and/or the expandable membrane 155 of the distal tip anchor 135 may be deployable into an expanded configuration upon the application of heat (e.g., temperatures between 35-40° C.). Alternatively or additionally, the expandable framework 140 and/or the expandable membrane 155 of the distal tip anchor may be deployable into an expanded configuration upon the application of an activation light. For example, the distal tip anchor 135 and all associated components may be constructed of materials that change their properties when exposed to certain frequencies of light, such that the distal tip anchor 135 and all associated components may expand, contract, retract, extend, or perform any combination or permutation of the aforementioned when under the application of predefined frequencies of light that are known to affect material properties.

Various methods of maintaining vessel patency within a patient are also contemplated. In this and other examples, and referring back to FIGS. 2A-2D, a method of maintaining vessel patency may include positioning an elongate shaft 115 of an introducer sheath 100 within a blood vessel V of a patient. The elongate shaft 115 may include a proximal end region 120, a distal end region 125, and a lumen 130 extending therebetween. The introducer sheath 100 may further include a hub 105 connected to the proximal end region 120 of the elongate shaft 115, a distal tip anchor 135 disposed proximate the distal end region 125, and an actuation assembly 110 operatively connected to the distal tip anchor 135. The method of this and other examples may further include delivering a medical device through the elongate shaft 115 of the introducer sheath 100 into the blood vessel V, actuating the actuation assembly 110 to deploy the distal tip anchor 135 from a collapsed configuration 160 to an expanded configuration, and positioning the distal tip anchor 135 against an inner wall of the blood vessel V with the distal tip anchor 135 in the expanded configuration while the medical device remains in the blood vessel V.

In this and other examples, methods of the present disclosure may further include advancing a proximal anchor 145 along a threaded exterior region 150 of the elongate shaft 115 to trap skin and/or body tissue S (e.g., epidermis, dermis and/or subcutaneous tissue) between the proximal anchor 145 and the distal tip anchor 135.

In this and other examples, methods of the present disclosure may further include breaking away the proximal end region 120 from the distal end region 125 by applying an outside force to a breakaway feature 175 disposed between the proximal end region 120 and the distal end region 125. Alternatively or additionally, the breakaway feature 175 may be present along any part or portion of the elongate shaft 115 of the introducer sheath 100, and may further include a plurality of breakaway features 175. The breakaway features 175 and breakaway action of the introducer sheath 100 of the present disclosure proves beneficial as a portion of the introducer sheath 100 (e.g., a portion of the distal end region 125) may remain stabilized within a vessel or similar anatomical structure while the proximal end region 120 or additional portions of components of the introducer sheath 100 may be taken away and disposed, allowing for greater room of operational freedom for a user or practitioner of the devices of the present disclosure by clearing away excess length of the introducer sheath 100 and associated components from the treatment site and treatment area.

Further, the breakaway feature 175 and/or plurality of breakaway features 175 may be any structure(s) that allows one or more portions of the elongate shaft 115 to be broken away from one or more other portions of the elongate shaft 115, typically along or proximate to the breakaway feature 175. By non-limiting examples, breakaway feature 175 may be a singular breakaway feature or include multiple breakaway features. Breakaway feature 175 may be in the form of a notch, a score mark, a groove, a perforation, a weaker material (i.e., material(s) weaker than adjacent and/or surrounding material(s)), a tearable material (i.e., a material that can be torn or broken away from adjacent and/or surrounding material), a splittable material, a heat-sensitive material, a cold-sensitive material, a tearable sheath (i.e. a sheath that can be torn or broken away from adjacent structure), a splittable sheath, a series of perforations, a series of notches, a series of score marks, a series of grooves, a series of weaker materials, a series of tearable materials, a series of splittable materials, a series of tearable sheaths, a series of splittable sheaths, or the like.

In this and other examples, methods of the present disclosure may include positioning the distal tip anchor 135 against an inner wall of the blood vessel V with the distal tip anchor 135 in the expanded configuration while the medical device remains within the blood vessel and may further include backing a length of the introducer sheath 100 out of the blood vessel V while the medical device remains within the blood vessel V and forming a seal against an interior wall of the blood vessel V with the distal tip anchor 135 in the expanded configuration. In other words, an approach to forming a vessel seal may include retracting a portion or a length of the introducer sheath 100 (after the introducer sheath 100 has been deployed within the vessel), and then deploying the distal tip anchor 135 by actuation of an actuation element 110, such that the distal tip anchor 135 and associated expandable framework 140 and/or expandable membrane 155 expands into an expanded configuration to form a seal against an interior wall of the blood vessel. Alternatively or additionally, the distal tip anchor 135, including the expandable framework 140 and/or expandable membrane 155, may expand into the expanded configuration upon being deployed outside the introducer sheath 100, or outside of the introducer sheath lumen 130 by virtue of being heat-set and/or radially biased outward in its material construction.

Various modifications and additions can be made to the exemplary embodiments discussed without departing from the scope of the present disclosure. For example, while the embodiments described above refer to particular features, the scope of the present disclosure also includes embodiments having different combinations of features and embodiments that do not include all of the above-described features.

What is claimed is:

1. An introducer sheath assembly comprising:
a hub;
an elongate shaft, wherein the elongate shaft includes a proximal end region connected to the hub, a distal end region, and a lumen extending therethrough;
a distal tip anchor disposed proximate the distal end region; the distal tip anchor having a collapsed configuration and an expanded configuration;
wherein the hub comprises an actuation element operatively connected to the distal tip anchor; wherein actuation of the actuation element deploys the distal tip anchor into the expanded configuration.

2. The introducer sheath assembly of claim 1, wherein the distal tip anchor is contained within the elongate shaft in a collapsed configuration.

3. The introducer sheath assembly of claim 1, wherein the distal tip anchor is collapsed along an exterior of the elongate shaft in the collapsed configuration.

4. The introducer sheath assembly of claim 1, wherein the distal tip anchor includes an expandable framework.

5. The introducer sheath assembly of claim 4, wherein the distal tip anchor further comprises a membrane affixed to the expandable framework.

6. The introducer sheath assembly of claim 4, wherein the expandable framework includes a spiral member configured to expand radially outward from a distal tip of the elongate shaft upon actuation of the actuation element.

7. The introducer sheath of claim 4, wherein the expandable framework includes a plurality of struts, wherein the plurality of struts are configured to expand radially outward from a distal tip of the elongate shaft upon actuation of the actuation element.

8. The introducer sheath of claim 4, wherein the expandable framework includes a pair of opposing beams, the pair of opposing beams configured to expand radially outward from the distal tip of the elongate shaft upon actuation of the actuation element.

9. The introducer sheath of claim 1, further comprising a proximal anchor advanceable along an exterior of the shaft.

10. The introducer sheath of claim 9, wherein the proximal anchor is advanceable along a threaded exterior region of the elongate shaft.

11. The introducer sheath of claim 9, wherein the proximal anchor is configured to be advanced distally along the exterior of the elongate shaft to trap body tissue between the proximal anchor and the distal tip anchor.

12. An introducer sheath assembly, comprising;
a hub;
an elongate shaft extending distally from the hub to a distal tip of the elongate shaft, the elongate shaft including a lumen extending therethrough;
a distal tip anchor comprising an expandable framework having a collapsed configuration when positioned within the lumen and an expanded configuration when deployed from the distal tip of the elongate shaft;
wherein the hub comprises an actuation element operatively connected to the distal tip anchor;
wherein actuation of the actuation element deploys the distal tip anchor into the expanded configuration.

13. The introducer sheath assembly of claim 11, further comprising a proximal anchor advanceable along an exterior of the elongate shaft to trap body tissue between the proximal anchor and the distal tip anchor.

14. The introducer sheath assembly of claim 13, wherein the proximal anchor is advanceable along a threaded exterior region of the elongate shaft.

15. A method of maintaining vessel patency within a patient, the method comprising:
positioning an elongate shaft of an introducer sheath within a blood vessel of a patient, the elongate shaft including a proximal end region, a distal end region, and a lumen extending therebetween; the introducer sheath further including a hub connected to the proximal end region of the elongate shaft, a distal tip anchor disposed proximate the distal end region, and an actuation assembly operatively connected to the distal tip anchor;
delivering a medical device through the elongate shaft of the introducer sheath into the blood vessel;
actuating the actuation assembly to deploy the distal tip anchor from a collapsed configuration to an expanded configuration;
positioning the distal tip anchor against an inner wall of the blood vessel with the distal tip anchor in the expanded configuration while the medical device remains within the blood vessel.

16. The method of claim 15, the method further comprising:
advancing a proximal anchor along a threaded exterior region of the elongate shaft to trap body tissue between the proximal anchor and the distal tip anchor.

17. The method of claim 15, the method further comprising:
breaking away the proximal end region from the distal end region by applying an outside force to a breakaway feature disposed between the proximal end region and the distal end region.

18. The method of claim 15, wherein the distal tip anchor further comprises an expandable framework.

19. The method of claim 18, wherein the distal tip anchor further comprises a membrane affixed to the expandable framework.

20. The method of claim 15, wherein positioning the distal tip anchor against an inner wall of the blood vessel with the distal tip anchor in the expanded configuration while the medical device remains within the blood vessel further comprises backing a length of the introducer sheath out of the blood vessel while the medical device remains within the blood vessel and forming a seal against an interior wall of the blood vessel with the distal tip anchor in the expanded configuration.
